(19) United States
(12) Patent Application Publication
(10) Pub. No.: US 2013/0014761 A1
(43) Pub. Date: Jan. 17, 2013

(54) ENDOTRACHEAL TUBE HAVING ONE OR MORE BLOCKING ELEMENTS, BLOCKING ELEMENTS, AND A METHOD OF USING BLOCKING ELEMENTS

(71) Applicants: Liron Elia, Kiryat-Ata (IL); Gavriel J. Iddan, Haifa (IL); Nir Lilach, Kfar Yehoshua (IL); Eliahu Eliachar, Haifa (IL)

(72) Inventors: Liron Elia, Kiryat-Ata (IL); Gavriel J. Iddan, Haifa (IL); Nir Lilach, Kfar Yehoshua (IL); Eliahu Eliachar, Haifa (IL)

(73) Assignee: ART Healthcare Ltd., Ashkelon (IL)

(21) Appl. No.: 13/635,949

(22) PCT Filed: Jan. 27, 2011
(86) PCT No.: PCT/IL11/00101

§ 371 Date: Sep. 19, 2012

Related U.S. Application Data
(60) Provisional application No. 61/340,622, filed on Mar. 22, 2010.
(60) Provisional application No. 61/365,399, filed on Jul. 19, 2010.

(51) Int. Cl. A61M 16/04

(52) U.S. Cl. 128/207.15

(57) ABSTRACT

An endotracheal device that comprises an endotracheal tube sized and shaped for being disposed within the trachea so that at least a distal segment thereof being placed in the windpipe lumen of a patient and at least one self expending element disposed around a peripheral surface of the endotracheal tube and having a first thickness in a compressed state and a second thickness in an expanded state, the at least one self expending element switching from the compressed state to the expanded state when absorbing moisture. The first thickness is thinner than the second thickness.

### FIELD AND BACKGROUND OF THE INVENTION

The present invention, in some embodiments thereof, relates to medical delivery tubes and, more particularly, but not exclusively, to endotracheal tubes and a method of using and producing thereof.

An endotracheal tube (also called an ET tube or ETT) is used in general anaesthesia, intensive care and emergency medicine for airway management, mechanical ventilation and as an alternative route for many drugs if an IV line cannot be established. The tube is inserted into a patient's trachea in order to ensure that the airway is not closed off and that air is able to reach the lungs. The endotracheal tube is regarded as the most reliable available method for protecting a patient's airway.

Ventilator-associated pneumonia (VAP) is a common complication which occurs when ETT is used; aspiration of bacteria colonized secretions across the endotracheal tube cuff into the lower airways is a major risk factor for VAP. Such aspiration occurs along longitudinal folds formed when the high-volume low-pressure endotracheal tube cuff is inflated in the trachea.

During the last years various solutions have been developed for reducing or avoiding VAP. For example, U.S. Patent Application No. 2009/0107510, filed on Oct. 29, 2007 describes a novel two-layer endotracheal tube (ETT) cuff for the prevention of pneumonia is disclosed. The disclosed two-layer ETT comprises a standard HVLP cuff covered with a second layer of elastomeric material with a sterile gel inserted between the layers. The two-layer cuff forms no folds when inflated in the trachea and prevents leakage, substantially reducing the risk for pneumonia attributable to standard ETT cuffs.

Another solution is described in U.S. Pat. No. 5,725,510, filed on Feb. 21, 1996 which describes an endotracheal tube with a collar. In order to avoid as far as possible the danger of a pulmonary infection caused by microbes introduced along the tube, at least one device with an antimicrobial action is fitted at one or more points on the outer surface of the tube. This device consists preferably of a piece of silver foil, vapor-deposited silver or a silver compound (silver salt), or may also be a length of tubing fitted in the tube.

### SUMMARY OF THE INVENTION

According to some embodiments of the present invention, there is provided an endotracheal device. The endotracheal device comprises an endotracheal tube sized and shaped for being disposed within the trachea so that at least a distal segment thereof being placed in the lumen of the windpipe of a patient and at least one self expending element disposed around a peripheral surface of the endotracheal tube and having a first thickness in a compressed state and a second thickness in an expanded state, the at least one self expending element switching from the compressed state to the expanded state when absorbing moisture. The first thickness is thinner than the second thickness.

Optionally, the at least one self expending element having a disc shaped structure around the endotracheal tube when in the expanded state.

Optionally, the at least one self expending element comprises at least one of compressed cellulose and Polyvinyl acetate (PVA).

Optionally, the second thickness is at least ten folds thicker than the first thickness.

Optionally, the at least one self expending element is at least partly soaked with a dissolvable material so as to allow the slowing down of its expending rate.

Optionally, the endotracheal device further comprises a suction unit for applying a suction force for drawing biological fluids accumulated in the trachea, above the at least one self expending element.

More optionally, the suction unit having a plug for transmitting the suction force from an external source to a space above the at least one self expending element, in proximity to the endotracheal tube.

More optionally, the suction unit having a mechanical valve for timing the applying, the mechanical valve being operated by the suction force.

More optionally, the endotracheal device further comprises at least one sensor for detecting at least one of a presence and an absence of biological fluids above the at least one self expending element, in proximity to the endotracheal tube, the suction unit being operated according to at least one of the presence and the absence.

More optionally, the endotracheal device further comprises a suction timing unit for timing the operation of the suction unit.

More optionally, the endotracheal device further comprises the suction timing unit having a mechanic valve for timing the applying.

More optionally, the endotracheal device further comprises the suction timing unit having a solenoid based valve for timing the applying.

More optionally, the endotracheal device further comprises the timing is performed in every preset period.

More optionally, the endotracheal device further comprises a suction indication unit for indicating whether the suction force is applied.

Optionally, the endotracheal tube is sized and shaped for passing via an incision in the trachea.

Optionally, at least one self expending element is circularly disposed around the peripheral surface.

More optionally, the endotracheal device further comprises a built in peristaltic pump for applying a suction force for drawing biological fluids accumulated above the at least one self expending element.

According to some embodiments of the present invention, there is provided a method of at least one of performing an endotracheal procedure. The method comprises providing an endotracheal tube having an inner lumen at least one self expending element disposed around a peripheral surface thereof, the at least one self expending element having a first thickness in a compressed state and a second thickness in an expanded state, the at least one self expending element switching from the compressed state to the expanded state when absorbing moisture, disposing the endotracheal tube within the trachea so that a distal segment thereof being in a trachea lumen of a patient, allowing the at least one self expending element to absorb biological fluids so as to change from the compressed state to the expanded state in the windpipe trachea lumen, and using the inner lumen for performing the endotracheal procedure.

Optionally, the endotracheal procedure is a member of a group consisting of a diagnostic procedure, a breathing procedure and a treatment of a trachea or the lungs, for example medicament injection.

According to some embodiments of the present invention, there is provided a blocking element of an endotracheal tube. The blocking element comprises a supporting member having an aperture sized for closely receiving an endotracheal tube and at least one self expending element coupled to the supporting member so as to be circularly disposed around a peripheral surface of the endotracheal tube. The at least one self expending element having a first thickness in a compressed state and a second thickness in an expanded state, the at least one self expending element switching from the compressed state to the expanded state when absorbing moisture.

According to some embodiments of the present invention, there is provided a endotracheal device that comprises an endotracheal tube sized and shaped for being disposed within the trachea so that at least a distal segment thereof being placed in the windpipe lumen of a patient and at least one flexible and absorbent element each disposed around a peripheral surface of the endotracheal tube so as to project outwardly and extend the cross section area thereof.

Optionally, the at least one flexible and flexible and absorbent element changes thickness when absorbing moisture.

Optionally, the at least one flexible and flexible and absorbent element is made of spongy material.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIG. 1A is a schematic illustration of a distal tip of an endotracheal device having an self expending element according to some embodiments of the present invention;

FIG. 1B is a blowup of the self expending element of FIG. 1A in an expended state, according to some embodiments of the present invention;

FIG. 1C is a blowup of the self expending element of FIG. 1A in a non expended state, according to some embodiments of the present invention;

FIG. 1D is a schematic illustration of the endotracheal device depicted in FIG. 1A when being disposed in the trachea, according to some embodiments of the present invention;

FIGS. 1E and 1F are schematic illustrations of an endotracheal tube with a fluid conducting tube to allow conducting water or another liquid solution toward the self expending element 102, according to some embodiments of the present invention;

FIG. 1G is a schematic illustration of the endotracheal device for tracheotomy or tracheostomy, according to some embodiments of the present invention;

FIG. 2 is a schematic illustration of the endotracheal device which is depicted in FIG. 1A with a suction unit for removing content accumulating between the endotracheal device and the trachea walls, according to some embodiments of the present invention;

FIG. 3 is a schematic illustration of an exemplary suction timing unit having valve disc controlled by a gear actuated by an actuating unit, according to some embodiments of the present invention;

FIG. 4 is a schematic illustration of an exemplary valve disc, according to some embodiments of the present invention;

FIGS. 5A and 5B are exemplary schematic illustrations of a solenoid valve controlled by the suction timing unit in open and closed states, according to some embodiments of the present invention;

FIGS. 5C and 5D are exemplary schematic illustrations of rotating valves, according to some embodiments of the present invention;

FIG. 6A is a schematic illustration of the endotracheal device that is depicted in FIG. 1A with a suction unit for removing content accumulating between the endotracheal device and the trachea walls where the suction unit has a suction timing unit which controls a vacuum regulator and a suction indication unit, according to some embodiments of the present invention;

FIG. 6B is a blowup of the suction indication unit which is depicted in FIG. 6A, according to some embodiments of the present invention;

FIG. 7A is a schematic illustration of the endotracheal device that is depicted in FIG. 1A with a suction unit which includes a peristaltic pump, according to some embodiments of the present invention;

FIGS. 7B and 7C depicts an endotracheal device with a sensor placed to read whether saliva, blood, food, and/or feeding fluids are accumulated in the trachea, according to some embodiments of the present invention; and

FIG. 8 is a flowchart of a method of in treatment and/or diagnosis, according to some embodiments of the present invention.

### DESCRIPTION OF EMBODIMENTS OF THE INVENTION

The present invention, in some embodiments thereof, relates to medical delivery tubes and, more particularly, but not exclusively, to endotracheal tubes and a method of using and producing thereof.

According to some embodiments of the present invention there are provided methods and endotracheal devices, such as a respiratory tube, a mechanical ventilation device having a respiratory tube, a lung probe conducting tube, and a medicament conducting tube. The tube has self expending elements for blocking body fluids or feeding fluids, such as saliva from passing to the lung during diagnosis, respiration, mechanical ventilation, and/or treatment of a patient via the trachea using one or more self expending elements, which are optionally expend when absorbing moisture. It should be noted that the term self expending element is used to described herein any flexible and absorbent element, such as flexible and absorbent element made of a spongy material or any element that changes thickness when absorbing moisture. Optionally, the endotracheal device includes endotracheal tube sized and shaped for being disposed within the trachea so that at least a distal segment thereof is placed in the windpipe lumen of a patient. The tube of the device, which may be a mechanical ventilation device, further includes one or more self expending elements disposed around, optionally a peripheral surface of the endotracheal tube and having a compressed state and an expanded state. The self expending elements optionally switches from the compressed state to the expanded state when absorbing biological fluids, such as saliva, food, feeding fluids, and blood or can already be disposed expended in body lumen, such as the trachea. The thickness in an expanded state is thicker than in a compressed state so that the gap between the inner walls of the trachea and the peripheral surface of the endotracheal tube at the respective cross section is sealed and/or substantially closed. Optionally the seal can be coated with bacteriostatic material for the prevention of infection in the trachea. Optionally, the expending elements are coated with lubricants. Optionally, a suction unit operated periodically and/or according to the reading of one or more sensors, is used for draining the saliva, blood, food, and/or feeding fluids from the trachea.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

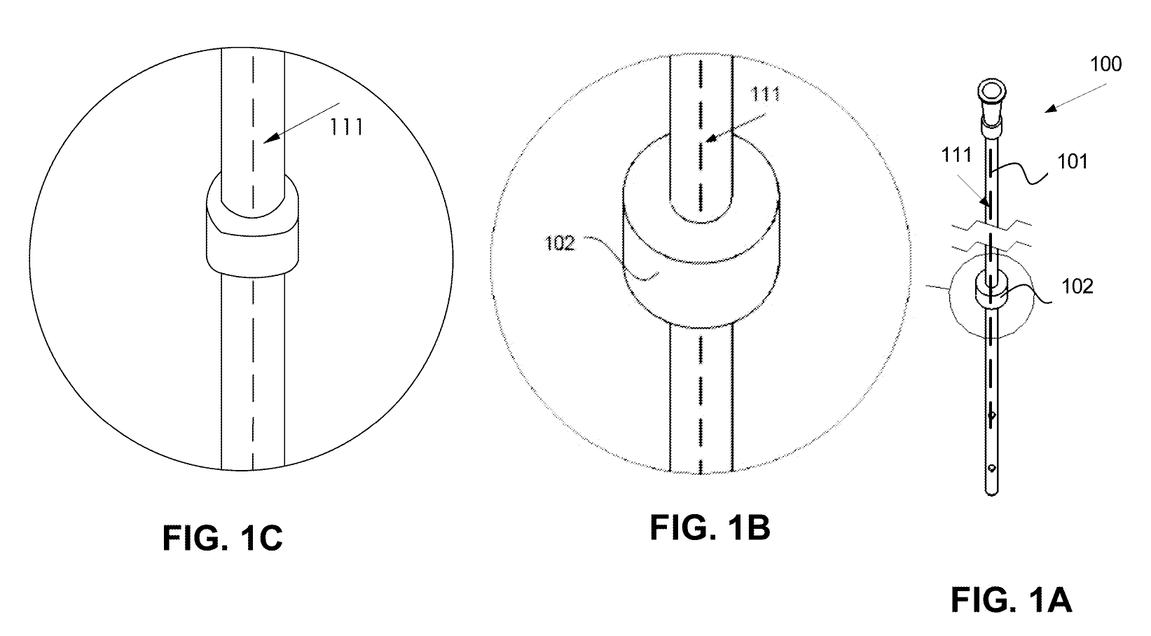

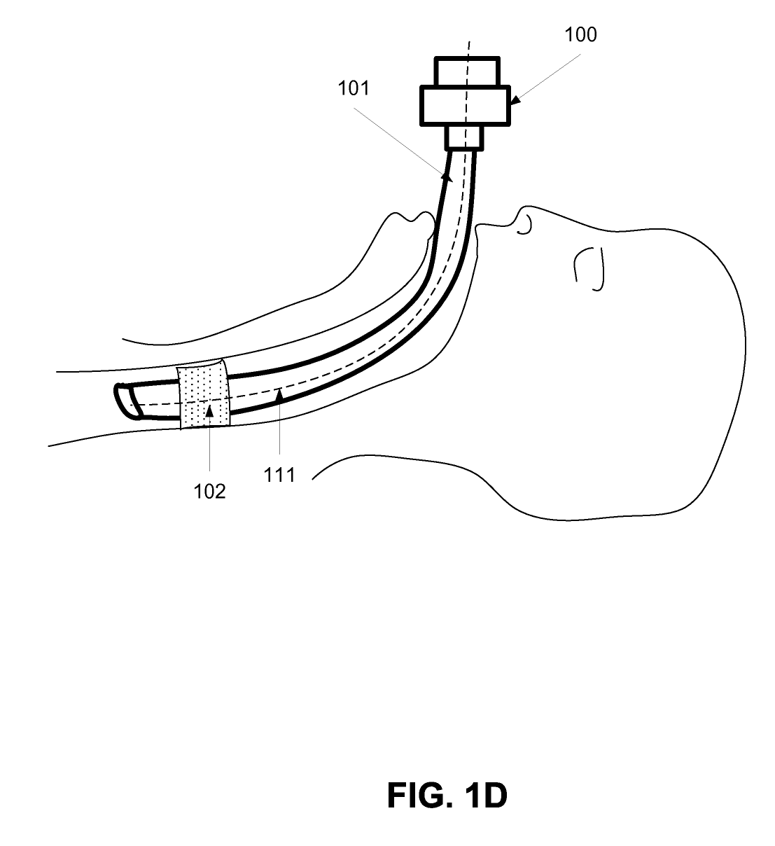

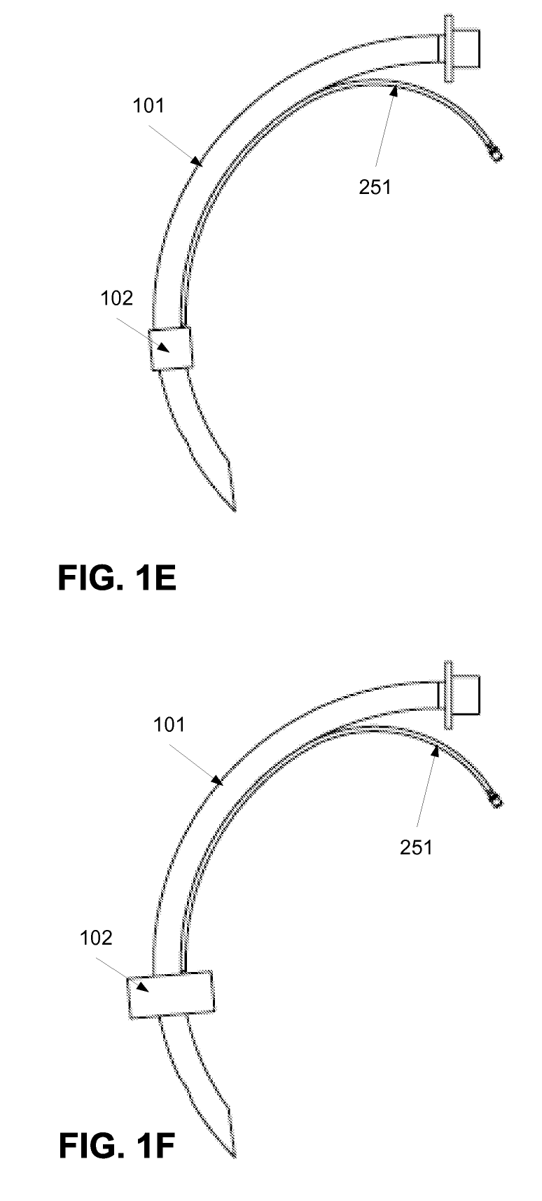

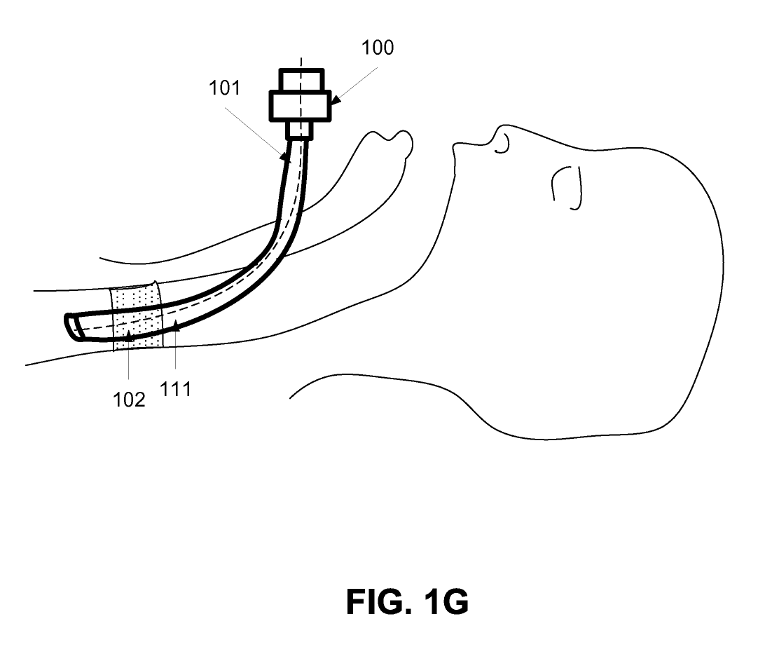

Reference is now made to FIG. 1A, which is a schematic illustration of a distal tip of an endotracheal device 100 having a self expending element 102 and to FIGS. 1B and 1C which are blowups of the self expending blocking element 102, for brevity referred to herein as a self expending element 102 respectively in an expended and non expended states, according to some embodiments of the present invention.

The endotracheal device 100 includes an endotracheal tube 101 having an inner lumen for respiration and/or delivering medications or for diagnostic purposes (probe). The endotracheal tube 101 is defined herein as any commonly used endotracheal tube, for example a respiratory tube of a mechanical ventilation system, a lung diagnosis catheter, and a medicament conducting tube. The endotracheal tube 101 may be used for guiding probes and/or sensors for lung or trachea diagnosis, for example imaging sensors borescope. The endotracheal tube 101 is sized and shaped for being disposed within the trachea so that a distal segment thereof is placed in the windpipe lumen of a patient. Optionally, the endotracheal tube 101 comprises a small diameter flexible tube preferably made of transparent plastic, such as polyvinyl Chloride or silicone. The length of the endotracheal tube 101 is adjusted to the size of the patient. For example, an endotracheal device for adult patients has an endotracheal tube 101 of more than 30 centimeter long and 1 cm diameter for and an endotracheal device for infants has an endotracheal tube 101 of more than 20 centimeter long and 0.5 cm diameter.

The endotracheal device 100 further comprises one or more self expending elements 102 placed to encircle, optionally substantially horizontality, an annular portion of the surface of the endotracheal tube 101, substantially perpendicularly to the main longitudinal axis 111 of the endotracheal tube 101. Each self expending element 102 may include one or more segments which encircle, at least substantially, a cross section of the endotracheal tube 101. For example a number of segments may be disposed around a common plane one to the side of the other and/or in parallel planes, one above the other.

In use, the self expending elements 102 are set to expand in the trachea, blocking saliva, blood, food, and/or feeding fluids from dripping into the lungs volume. The self expending elements 102 have at least two states, a compressed state and an uncompressed expanded state. When the self expending element(s) 102 are in a compressed state, the endotracheal device 100 may be guided via tubular lumens having a limited diameter, such as the trachea, without applying damaging pressure on the inner walls. However, when the self expending element(s) 102 are in an expanded state, their diameter increases and a flexible is created for the tubular lumen.

Optionally, the self expending element 102 is made of a biocompatible material such as crystal violet—A dye derived from gentian violet that is used as a general biological stain, an acid-base indicator, and an agent against infection by bacteria, fungi, pinworms, and other parasites. The biocompatible material is optionally porous, which expands when it absorbs biological fluids, for example the material is a spongy material, such as compressed cellulose and Polyvinyl acetate (PVA) or polyvinyl formal (PVF) that is manufactured from PVA by reaction with butyraldehyde. Optionally, the self expending element 102 is between about 0.2 mm thick and about 2 mm thick in a compressed state and about ten time thicker in an expanded state, for example when exposed to moist or biological fluids. Each self expending element 102 is optionally shaped as a tube and coupled on a peripheral surface of the endotracheal tube 101 so that expands the diameter at a certain cross section thereof. In such an embodiment, the compressed state is achieved when the porous martial is in a non absorbed state and the expanded state is achieved when the porous martial is absorbed with biological fluids.

The resulting shape of the self expending element 102 in an expended state approximates a tube or a cylindrical roll, expanded in size with respect to its compressed, non-absorbed state. Optionally, the self expending element 102 is comprises of a number of annular layers which are appended, one on top of the other. Different layers may have different expansion factor when exposed to biological fluids.

In use, at least a portion of the endotracheal device 100 is inserted through the nasal or oral cavity, passing through at least part of the trachea and terminating in the windpipe lumen. For example, when the endotracheal device 100 is a respiratory endotracheal device, the placing of the distal end of the endotracheal tube in the windpipe lumen allows direct ventilation to the lungs, via the inner lumen of the endotracheal tube 101.

The self expending element 102, which is optionally placed at the distal segment of the endotracheal device 100, functions as a seal, for example as shown at FIG. 1D. When saliva, blood, food, and/or feeding fluids drops toward the lungs, the self expending element 102 in the expanded state seals, or substantially closes, the -trachea passage.

Optionally, the self expending element 102 is wetted before the disposing of the device 100 in the trachea so as to reduce it's rigidify and/or to reduce its expansions time.

In use, the expanded state, the self expending element 102 fills the gap between the endotracheal tube 101 and the esophageal walls, preventing from some or all of the saliva, blood, food, and/or feeding fluids to pass from the trachea to the lungs. Such a self expending element 102 is passive, allowing sealing or substantially closing off the trachea passage without using actuating means.

According to some embodiments of the present invention, the self expending element 102 is at least partly soaked with a dissolvable material so as to reduce its expansion rate, or any other polymeric material to be used as a sleeve. For example, a gelatin base material or any other dissolvable material that withholds the self expending element 102 from absorbing the biological fluids when placing the endotracheal tube 101 in the trachea of the patient is applied. In such a manner, the self expending element 102 remains in a compressed state for a locating period in which the user can easily locating the endotracheal tube 101 in the trachea. The gelatin base material dissolves after a couple of minutes when the endotracheal tube 101 is in place in the trachea 103. During the locating period the operator introduces the endotracheal tube easily and comfortably with no excessive friction. After the endotracheal tube 101 is in proper position and the compressed self expending element 102 is in the lower portion of the trachea, the gelatin dissolves and the self expending element 102 absorbs the, blood, and/or feeding fluids from the surrounding and as an outcome expends.

It should be noted when the self expending element 102 absorbs fluids, it soften and becomes more elastic. This facilitates the removing thereof.

Optionally, as shown at FIGS. 1E and 1F depict a fluid conducting tube 251 which is attached along the endotracheal tube 101 to allow conducting water or another liquid solution toward the self expending element 102. In such a manner, the expansion of the self expending element 102 may be catalyzed. FIG. 1E depicts the self expending element 102 before the exposure to the conducted water and FIG. 1F depicts the self expending element 102 after the exposure to the conducted water.

It should be noted that the endotracheal device 100 may be adjusted for tracheotomy and/or tracheostomy, as shown at FIG. 1G. In such an embodiment, the endotracheal tube 101 and the self expending element 102 are sized and shaped for being placed in a incision, such as a curvilinear skin incision in the trachea optionally along the relaxed skin tension lines (RSTL) between sternal notch and cricoid cartilage. In such embodiments the endotracheal device 100 opens a direct airway through the incision.

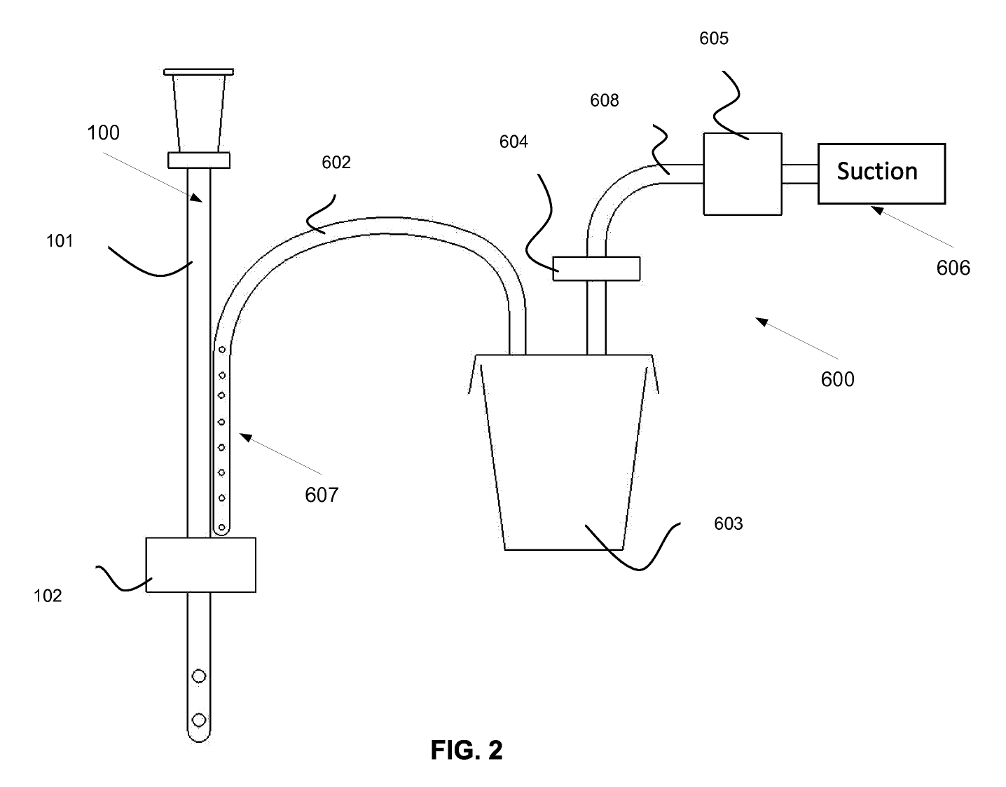

Reference is now made to FIG. 2, which is a schematic illustration of the endotracheal device 100 which is depicted in FIG. 1A with a suction unit 600 for removing content accumulating between the endotracheal device 100 and the trachea walls, above the self expending element 102, according to some embodiments of the present invention. The suction unit 600 may be set as a separate unit, for example provided part of a kit and/or as a separate product and/or part of the endotracheal device 100, for example attached or detachably attached to the endotracheal tube 101. The suction unit 600 includes a suction tube 602 having a distal segment with one or more apertures for suction, for example as shown at 607. The suction tube 602 is set to connect operatively to a suction source 606, such as a standard operating room vacuum system or a pump, for example a small scale piston pump or a peristaltic pump. The suction source 606 may be manual, for example syringe-type plunger (not shown). This suction unit 600 allows draining the, blood, food, and/or feeding fluids which accumulates, when the endotracheal device 100 is inserted into the trachea of the patient, between the exterior walls of the endotracheal tube 101 and the trachea walls for example every predefined period, manually upon request, and/or upon a signal received from one or more sensors, and the like. Optionally, as depicted in FIG. 2, the suction unit 600 includes a draining tank 603, a filter 604, and/or a suction timing unit 605, connected to the draining tank 603 via a suction source tube 608. In use, the drained biological fluids are accumulated in the draining tank 603. The draining tank 603 is optionally detachably connected to the suction unit 600. In such a manner, the draining tank 603 may be emptied when full and/or from time to time. Optionally, a conduit is connected to the draining tank 603, facilitating a continuous emptying thereof. The filter 604 filters the fluids which are drained toward the suction source 606, preventing from the plugging thereof by the drained, blood, food, and/or feeding fluids. Optionally, the suction timing unit 605 is set to open a valve. The opening of the valve allows the applying of a suction force that drains, or substantially drains, the accumulated saliva, blood, food, and/or feeding fluids. The suction timing unit 605 may be set to open the valve every predefined idle period, for example every minute, 5 minutes, 10 minutes, 60 minutes, 120 minutes, and/or any intermediate or longer periods for a predefined suction period, for example 10 seconds, 30 seconds, 1 minute, 5 minutes and/or any intermediate or shorter periods.

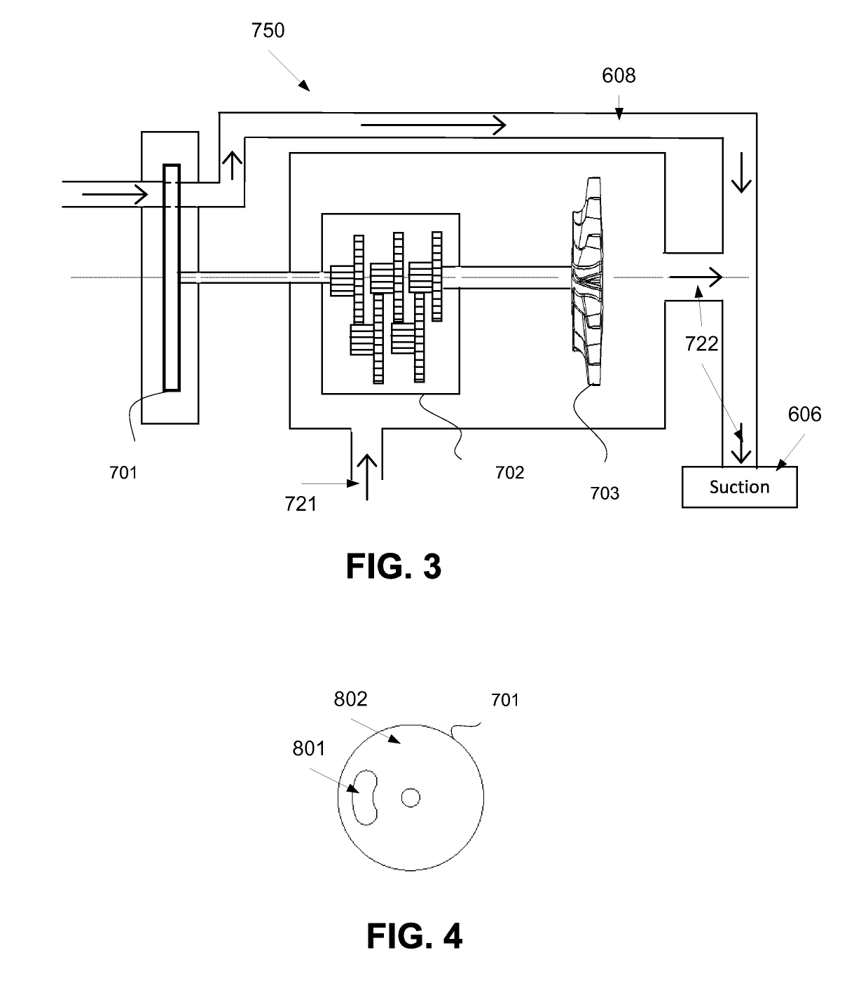

FIG. 3 is a schematic illustration of an exemplary suction timing unit 750 having valve disc 701 controlled by a gear 702 actuated by an actuating unit 703, such a turbine 704. In this embodiment the turbine is automatically actuated by the suction force which is applied from the suction source 606. The turbine actuating force route is indicated by numerals 721, 722.

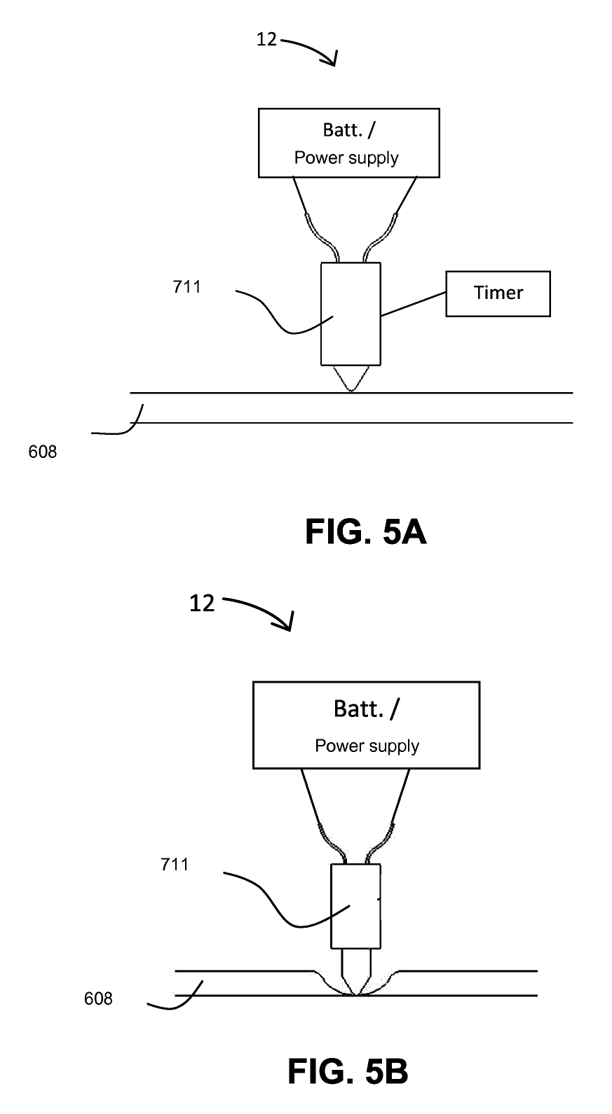

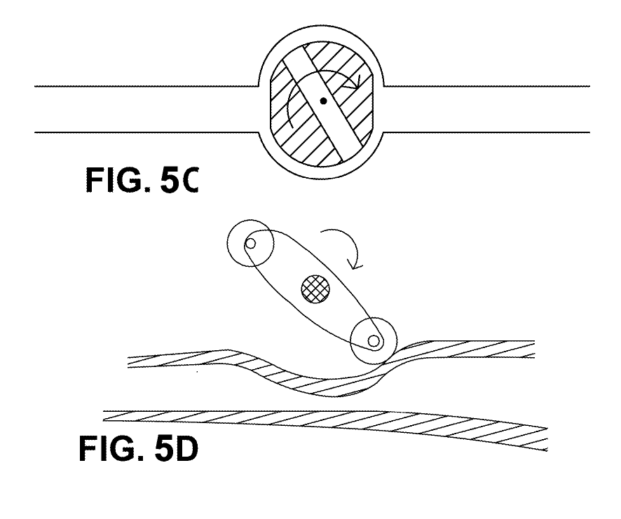

The valve disc 701 is placed in a cross section of a suction force conduit 705 which connects between the tip of the suction source tube 608 and the suction source 606. The gear 702 is set to rotate the valve disc 701, which is optionally shaped with a suction force opening segment 801 and a blocking surface segment 802, as depicted in FIG. 4, in a preset pace. When the suction force opening segment 801 is placed in the cross section of the suction source tube 608, suction force is applied. The preset pace assures a certain predefined idle, namely when the blocking surface segment 802 is placed in the cross section of the suction source tube 608 and a certain predefined suction period, namely when the suction force opening segment 801 is placed in the cross section of the suction source tube 608. FIGS. 5A and 5B are exemplary schematic illustrations of a solenoid valve 711 which is controlled by the suction timing unit 605 in open and closed states, according to some embodiments of the present invention. The solenoid valve 711 is placed to block a cross section of the suction source tube 608 which connects between the tip of the suction tube 602 and the suction source 606. For blocking the suction force, the solenoid applies pressure on the suction source tube 608. For facilitating the suction force, the pressure is released.

FIGS. 5C and 5D are exemplary schematic illustrations of rotating valves. In FIG. 5C a plate which rotates in the fluid tube regulates the suction and in FIG. 5D a rotating lever having two wheels attached to its lateral sides is set to apply interchangeably pressure on the tube, moving it between open and closed states, according to some embodiments of the present invention.

Additionally or alternatively, the suction force may be applied according to the reading of one or more sensors, such as impedance sensors. In such an embodiment, the suction timing unit 605 receives the reading of the impedance sensors and operates a suction force valve and/or the suction source 606 accordingly. The suction timing unit 605 may be operated by batteries and/or external AC power. Optionally, the suction timing unit 605 has a plug adapted to the suction source 606 socket of a hospital and/or an ambulance and/or a hospitalization facility. Drainage may be done manually with a syringe connected to the tube for example.

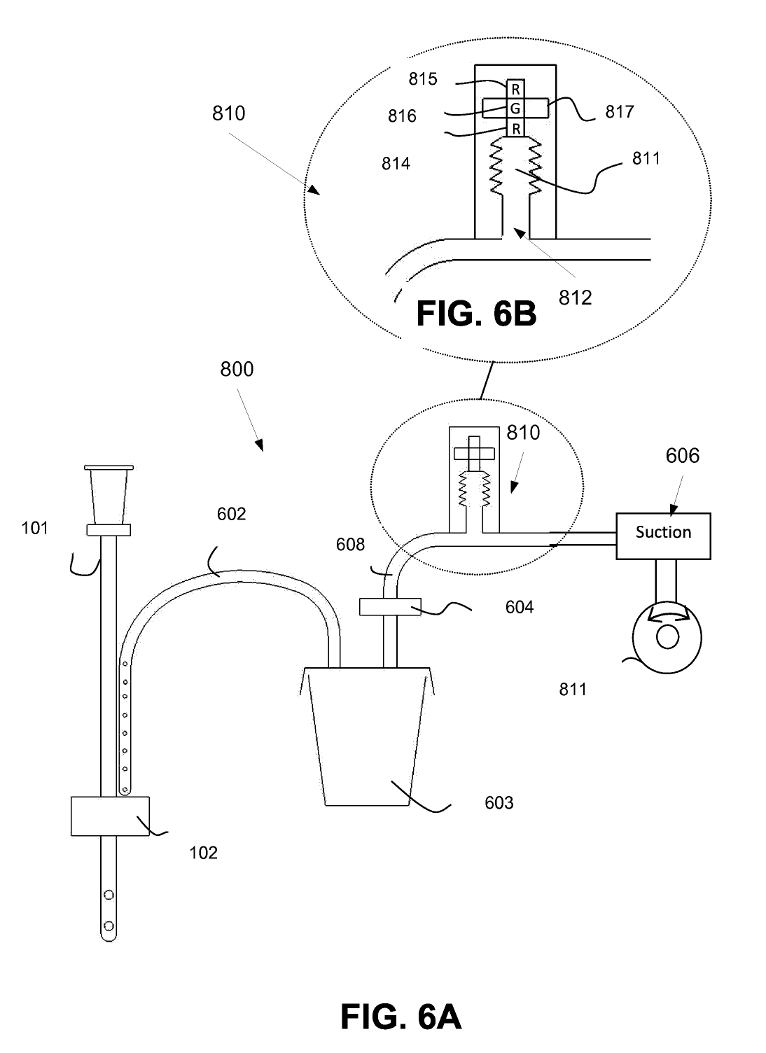

Reference is now made to FIG. 6A, which is a schematic illustration of the endotracheal device 100 that is depicted in FIG. 1A with another suction unit 800 for removing content accumulating between the endotracheal device 100 and the trachea walls, above the self expending element 102, according to some embodiments of the present invention. In this embodiment, the suction unit 800 has a suction timing unit which controls a vacuum regulator 811 to regulate the suction power according to readings of one or more sensors and/or periodically, for example as described above. Optionally, the suction unit 800 further includes a suction indication unit 810, optionally mechanical, for example as depicted in FIG. 6A and in the blowup of the suction indication unit 810 which is depicted in FIG. 6B. The suction indication unit 810 indicates whether a suction force is applied by the suction unit 800 or not. For example, in the embodiment depicted in FIG. 6B, a lower tip 812 of bellow 811 is connected to the suction force conduit 705 of the suction unit 800. The upper tip 813 of the bellow 811 is connected to a sign 814 elevated or lowered according to the suction force in the suction force conduit 608. The change in the elevation can be seen from an indication window 817, optionally made of a transport polymeric material. Optionally, the sign is interchangeably colored with different colors, for example red 815 and green 816, so that while one color indicates a low pressure, the other indicates a high pressure. Additionally or alternatively, a solid state may can be applied for indicates a high pressure.

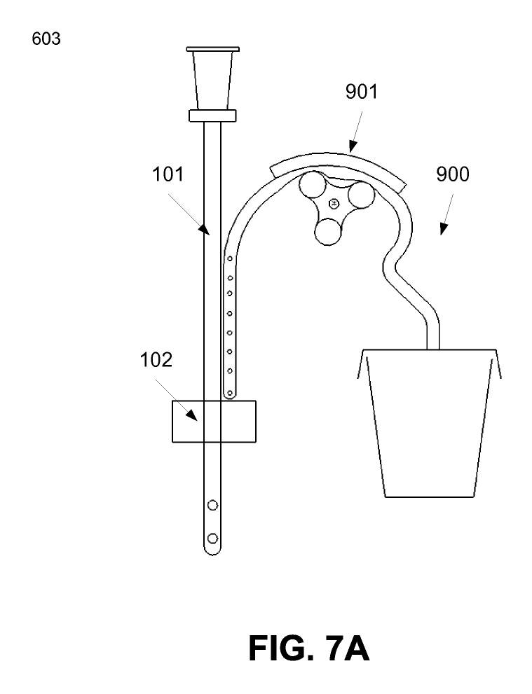

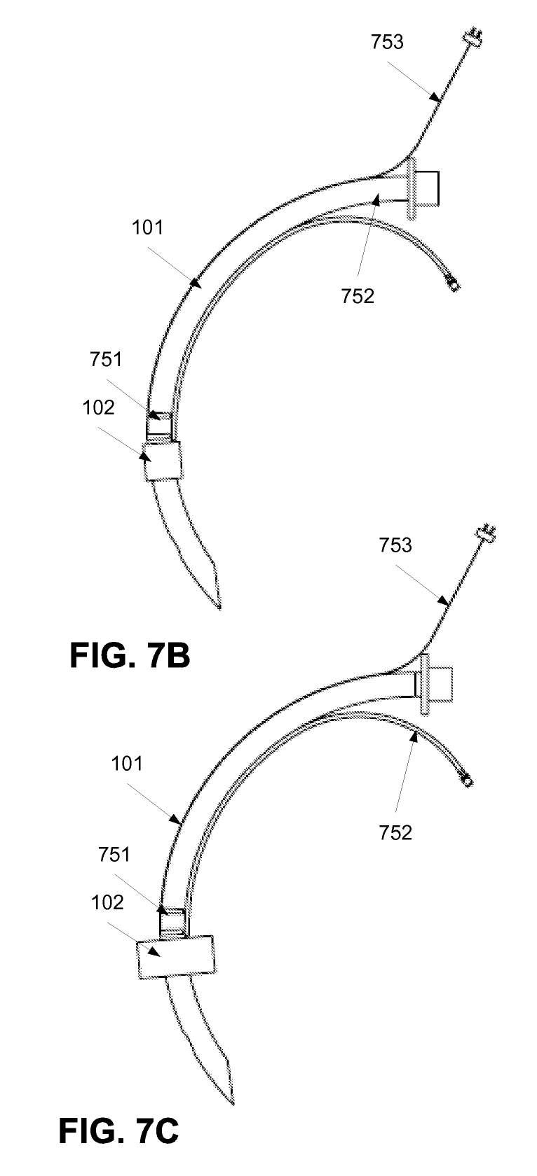

Reference is now made to FIG. 7, which is a schematic illustration of the endotracheal device 100 that is depicted in FIG. 1A with another suction unit 900 for removing content accumulating between the endotracheal device 100 and the trachea walls, above the self expending element 102, according to some embodiments of the present invention. The suction unit 900 includes a peristaltic pump 901 which changes, in use, the suction force applied in the trachea. The peristaltic pump 901 is optionally built in and may be operated by the suction timing unit 605, periodically and/or according to the readings of sensors and/or upon request, for example when the user operates it. For example, FIG. 7B depicts a sensor 751 which is placed to read whether saliva, blood, food, and/or feeding fluids are accumulated in the trachea. The sensor 751 is connected to a conductive line 753 so as to forward its reading the suction unit. A tube 752 for suction is also depicted herein., FIG. 7C depicts the self expending element 102 in an expended mode. Optionally, the conductive line 753 is connected to a controller which controls or regulates the suction of, blood, food, and/or feeding fluid according to readings of the sensor 753.

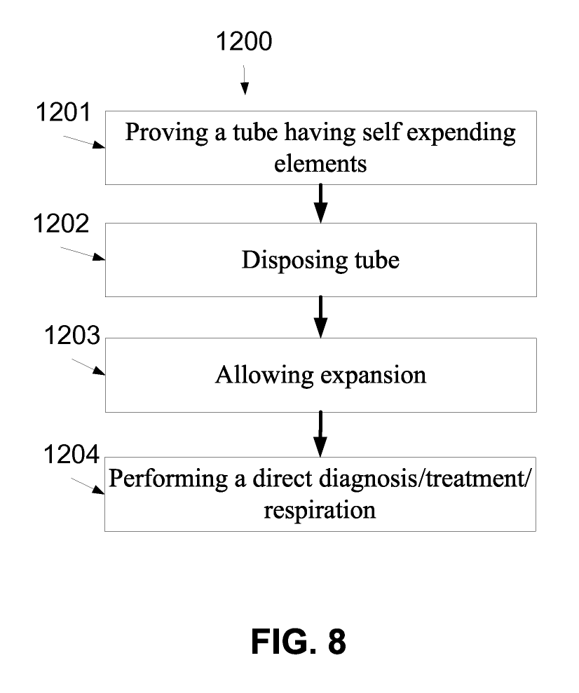

Reference is now also made to FIG. 8, which is a flowchart of a method 1200 of respiration, treatment and/or diagnosis, according to some embodiments of the present invention. First, as shown at 1201, an endotracheal tube, such as 101, having an inner lumen is provided. The endotracheal tube may have an inner lumen for respiration or for delivering medication and/or one or more diagnostic sensors, such as pH sensors, image sensors, fluid sensors, and the like.

One or more self expending elements 102, as shown at FIG. 1A, are circularly coupled to the peripheral surface of the endotracheal tube 101. The self expending elements 102 are in a compress state, for example as described above. For example, the self expending elements 102 are made of PVA which is soaked with gelatin based material for decrease the biological fluid absorption rate. Optionally, the self expending elements 102 is covered with lubricants to facilitate the positioning of the endotracheal tube 101 in the trachea.

Now, as shown at 1202, the endotracheal tube is disposed within the trachea so that a distal end thereof is in the windpipe lumen of a patient, for example as shown at FIG. 1D. This allows the self expending elements 102 to expend, for example as described above and shown at 1203. The expansion forms an annular element around the endotracheal tube 101 that seals or substantially closes the trachea, for example as described above. As described above, the self expending element 102 is optionally located above the lungs and expands to block saliva, blood, food, and/or feeding fluids by sealing or substantially closing off the -trachea passage. Now, as shown at 1204, the endotracheal tube 101 may be used for directly performing a respiration and/or a treatment in the trachea lumen or the lungs. For example, the endotracheal tube 101 is a respiration tube that is used in general anaesthesia, intensive care and emergency medicine for airway management and/or for delivering medications via said inner lumen.

It is expected that during the life of a patent maturing from this application many relevant devices and methods will be developed and the scope of the term sensor is intended to include all such new technologies a priori.

As used herein the term “about” refers to ±10.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An endotracheal device, comprising:
an endotracheal tube sized and shaped for being disposed within the trachea so that at least a distal segment thereof being placed in the windpipe lumen of a patient; and
at least one self expending element disposed around a peripheral surface of said endotracheal tube at said distal segment and having a first thickness in a compressed state and a second thickness in an expanded state, said at least one self expending element switching from said compressed state to said expanded state when absorbing moisture;
wherein said first thickness is thinner than said second thickness.

2. The endotracheal device of claim 1, wherein said at least one self expending element having a disc shaped structure around said endotracheal tube when in said expanded state.

3. The endotracheal device of claim 1, wherein said at least one self expending element comprises at least one of compressed cellulose and Polyvinyl acetate (PVA).

4. The endotracheal device of claim 1, wherein the said second thickness is at least ten folds thicker than said first thickness.

5. The endotracheal device of claim 1, wherein said at least one self expending element is at least partly soaked with a dissolvable material.

6. The endotracheal device of claim 1, wherein said at least one self expending element having at least one conduit for conducting biological fluids from the top of said at least one self expending element to the bottom of said at least one self expending element.

7. The endotracheal device of claim 6, further comprising at least one conduit closing element having a closed state for substantially sealing said at least one conduit and an open state for substantially unsealing said at least one conduit.

8. The endotracheal device of claim 7, wherein said at least one conduit closing element having at least one flap, each said flap being swingably coupled on a peripheral surface of said endotracheal tube so that said at least one flap swings relative to said endotracheal tube, said at least one flap swinging between said closed state and said open state.

9. The endotracheal device of claim 1, wherein said endotracheal tube having at least one of a recess and a conduit for conducting biological fluids from the top of said at least one self expending element to the bottom of said at least one self expending element.

10. The endotracheal device of claim 9, further comprising a unidirectional valve disposed in the lumen of said at least one of said recess and said conduit.

11. The endotracheal device of claim 1, further comprising a suction unit for applying a suction force for drawing biological fluids accumulated above said at least one self expending element.

12. The endotracheal device of claim 11, wherein said suction unit having a plug for transmitting said suction force from an external source to a space above said at least one self expending element, in proximity to said endotracheal tube.

13. The endotracheal device of claim 12, wherein said suction unit having a mechanical valve for timing said applying, said mechanical valve being operated by said suction force.

14. The endotracheal device of claim 11, further comprising at least one sensor for detecting at least one of a presence and an absence of biological fluids above said at least one self expending element, in proximity to said endotracheal tube, said suction unit being operated according to at least one of said presence and said absence.

15. The endotracheal device of claim 11, further comprising a suction timing unit for timing the operation of said suction unit.

16. The endotracheal device of claim 15, wherein said suction timing unit having a mechanic valve for timing said applying.

17. The endotracheal device of claim 15, wherein said suction timing unit having a solenoid based valve for timing said applying.

18. The endotracheal device of claim 15, wherein said timing is performed in every preset period.

19. The endotracheal device of claim 11, further comprising a suction indication unit for indicating whether said suction force is applied.

20. The endotracheal device of claim 1, wherein at least one self expending element is circularly disposed around said peripheral surface.

21. The endotracheal device of claim 1, further comprising a built in peristaltic pump for applying a suction force for drawing biological fluids accumulated above said at least one self expending element.

22. The endotracheal device of claim 1, wherein said endotracheal tube is sized and shaped for passing via an incision in said trachea.

23. A method of at least one of performing an endotracheal procedure, comprising:
providing an endotracheal tube having an inner lumen at least one self expending element disposed around a peripheral surface at a distal segment thereof, said at least one self expending element having a first thickness in a compressed state and a second thickness in an expanded state, said at least one self expending element switching from said compressed state to said expanded state when absorbing moisture;
disposing said endotracheal tube within the trachea so that said distal segment being in a trachea lumen of a patient;
allowing said at least one self expending element to absorb biological fluids so as to change from said compressed state to said expanded state in said trachea lumen; and
using said inner lumen for performing the endotracheal procedure.

24. The method of claim 23, wherein said endotracheal procedure is a member of a group consisting of a diagnostic procedure, a respiratory procedure and a treatment of a windpipe lumen.

25. A blocking element of an endotracheal tube, comprising:
a supporting member having an aperture sized for closely receiving an endotracheal tube sized and shaped for being disposed within the trachea so that at least a distal segment thereof being placed in the windpipe lumen of a patient; and
at least one self expending element coupled to said supporting member so as to be circularly disposed around a peripheral surface of said distal end, said at least one self expending element having a first thickness in a compressed state and a second thickness in an expanded state, said at least one self expending element switching from said compressed state to said expanded state when absorbing moisture.

26. An endotracheal device, comprising:
an endotracheal tube sized and shaped for being disposed within the trachea so that at least a distal segment thereof being placed in the windpipe lumen of a patient; and
at least one flexible and absorbent element each disposed around a peripheral surface of said distal segment so as to project outwardly and extend the cross section area thereof.

27. The endotracheal device of claim 26, wherein said at least one flexible and flexible and absorbent element changes thickness when absorbing moisture.

28. The endotracheal device of claim 26, wherein said at least one flexible and flexible and absorbent element is made of spongy material.
